(19) United States
(12) Patent Application Publication
(10) Pub. No.: US 2014/0080850 A1
(43) Pub. Date: Mar. 20, 2014

(54) PHARMACEUTICAL COMPOSITION COMPRISING AN OMEGA-3 FATTY ACID AND A HYDROXY-DERIVATIVE OF A STATIN AND METHODS OF USING SAME

(71) Applicant: Amarin Pharmaceuticals Ireland Limited, Dublin (IE)

(72) Inventor: Richard Preston Mason, Bedminister, NJ (US)

(21) Appl. No.: 13/908,911

(22) Filed: Jun. 3, 2013

Related U.S. Application Data
(60) Provisional application No. 61/655,879, filed on Jun. 5, 2012.

(51) Int. Cl. A61K 31/505; A61K 31/202; A61K 31/366; A61K 31/40

(52) U.S. Cl. CPC A61K 31/505; A61K 31/40; A61K 31/202; A61K 31/366
USPC 514/275; 514/423; 514/460

(57) ABSTRACT

In various embodiments, the present invention provides compositions and methods for treating and/or preventing a cardiovascular-related disease in subject in need thereof.

### CROSS-REFERENCE TO RELATED APPLICATION(S)

This application claims priority to U.S. provisional patent application Ser. No. 61/655,879, filed on Jun. 5, 2012, the entire contents of which are incorporated herein by reference.

### BACKGROUND

Cardiovascular disease is one of the leading causes of death in the United States and most European countries. It is estimated that over 70 million people in the United States alone suffer from a cardiovascular disease or disorder including but not limited to high blood pressure, coronary heart disease, dislipidemia, congestive heart failure and stroke. A need exists for improved treatments for cardiovascular-related diseases and disorders.

### SUMMARY

In various embodiments, the present invention provides pharmaceutical compositions and methods of using such compositions to treat and/or prevent cardiovascular-related diseases. In one embodiment, a pharmaceutical composition comprising a statin or derivative of a statin, for example a hydroxy-derivative of a statin, or a pharmaceutically acceptable salt thereof and an omega-3 fatty acid and is provided. The term “hydroxy-derivative of a statin” herein refers to a parent statin compound (i.e. known class of HMG-CoA reductase inhibitors) having at least one hydroxy substituent group. In one embodiment, a hydroxyl group is attached to a phenyl ring of the parent statin.

In another embodiment, a pharmaceutical composition comprising a hydroxy-derivative of a statin or a pharmaceutically acceptable salt thereof and an oil comprising an omega-3 fatty acid is provided. In a related embodiment, the oil comprises at least 95% by weight eicosapentaenoic acid or derivative thereof, for example ethyl eicosapentaenoate.

In various embodiments, the hydroxy-derivative of a statin is selected from a hydroxy-derivative of atorvastatin, mevastatin, pitavastatin, pravastatin, rosuvastatin, fluvastatin, simvastatin, lovastatin, cerivastatin and pharmaceutically acceptable salts thereof.

In other embodiments, the hydroxy-derivative of a statin is selected from ortho or para hydroxy-atorvastatin, p-hydroxy atorvastatin calcium, p-hydroxy atorvastatin disodium, o-hydroxy atorvastatin calcium, o-hydroxy atorvastatin lactone, o-hydroxy atorvastatin-d5 calcium, o-hydroxy atorvastatin-d5 disodium, o-hydroxy atorvastatin-d5 lactone, 2-hydroxy atorvastatin bisodium, p-hydroxy atorvastatin lactone, p-hydroxy atorvastatin-d5 calcium, p-hydroxy atorvastatin-d5 lactone, and 4-hydroxy atorvastatin bisodium.

In still other embodiments, the oil comprises one or more of: (a) about 0.2% to about 0.5% by weight ethyl octadecatetraenoate, (b) about 0.05% to about 0.20% by weight ethyl nonaecapentaenoate, (c) about 0.2% to about 3% by weight ethyl arachidonate, (d) about 0.3% to about 0.5% by weight ethyl eicosatetraenoate, (e) about 0.8% to about 0.25% by weight ethyl heneicosapentaenoate, (f) about 0.02% to about 0.1% by weight ethyl 17E-icosapentaenoate, (g) about 0.02% to about 0.1% by weight ethyl 5-icosapentanoate, (h) about 0.01% to about 0.15% by weight ethyl 5E,8E-icosapentaenoate, (i) about 0.01% to about 0.15% by weight ethyl 8E,11E-icosapentaenoate, (j) about 0.01% to about 0.15% by weight ethyl 5E,14E-icosapentaenoate, (k) about 0.01% to about 0.15% by weight ethyl 5E,8E,11E,17E-icosapentaenoate, (l) no amount or substantially no amount of ethyl icosahexaenoate, (m) no amount or substantially no amount of ethyl 11Z-eicosenoate, (n) no amount or substantially no amount of ethyl docosahexaenoic acid, and/or (o) about 0.02% to about 0.1% ethyl nonadecapentaenoate.

In some embodiments, a pharmaceutical composition comprising a hydroxy-derivative of a statin or a pharmaceutically acceptable salt thereof and an oil comprising an omega-3 fatty acid is provided in which a molar ratio of the omega-3 fatty acid to the hydroxy-derivative of the statin is about 1:1 to about 20:1. In related embodiments, the molar ratio of the omega-3 fatty acid to the hydroxy-derivative of the statin is about 10:1.

In yet another embodiment, the invention provides a method of treating a cardiovascular-related disease in a subject in need thereof comprising administering a composition as described herein to the subject. In one embodiment, the cardiovascular-related disease is artherosclerosis.

These and other embodiments of the present invention will be disclosed in further detail herein below.

### BRIEF DESCRIPTION OF THE DRAWINGS

FIG. 1 shows effects of EPA, DHA and EPA/DHA, in combination with atorvastatin, atorvastatin o-hydroxy metabolite, simvastatin or rosuvastatin, on membrane lipid peroxidation.

FIG. 2 shows effects of EPA in combination with atorvastatin, atorvastatin o-hydroxy metabolite, simvastatin or rosuvastatin, on membrane lipid peroxidation.

FIG. 3 shows effects of DHA in combination with atorvastatin, atorvastatin o-hydroxy metabolite, simvastatin or rosuvastatin, on membrane lipid peroxidation.

FIG. 4 shows effects of EPA/DHA in combination with atorvastatin, atorvastatin o-hydroxy metabolite, simvastatin or rosuvastatin, on membrane lipid peroxidation.

FIG. 5 shows effects of atorvastatin, atorvastatin o-hydroxy metabolite, simvastatin or rosuvastatin, in combination with EPA, DHA or EPA/DHA on membrane lipid peroxidation.

FIG. 6 shows effects of atorvastatin, in combination with EPA, DHA or EPA/DHA, on membrane lipid peroxidation.

FIG. 7 shows effects of atorvastatin o-hydroxy metabolite in combination with EPA, DHA or EPA/DHA, on membrane lipid peroxidation.

FIG. 8 shows effects of simvastatin, in combination with EPA, DHA or EPA/DHA, on membrane lipid peroxidation.

FIG. 9 shows effects of rosuvastatin in combination with EPA, DHA or EPA/DHA, on membrane lipid peroxidation.

FIG. 10 shows the separate and combined effects of EPA, atorvastatin (Atorv), atorvastatin metabolite (ATM), and VAS2780 (VAS) on calcium-stimulated NO release from human umbilical vein endothelial cells exposed to oxidized LDL (oxLDL).

FIG. 11 shows the separate and combined effects of EPA, atorvastatin (Atorv), atorvastatin metabolite (ATM), and VAS2780 (VAS) on calcium-stimulated ONOO⁻ release from human umbilical vein endothelial cells exposed to oxidized LDL (oxLDL).

FIG. 12 shows the separate and combined effects of EPA, atorvastatin (Atorv), atorvastatin metabolite (ATM), and VAS2780 (VAS) on the ratio of NO to ONOO⁻ release from human umbilical vein endothelial cells exposed to oxidized LDL (oxLDL).

FIG. 13 shows the separate and combined effects of EPA, rosuvastatin (Rosuv), and VAS2780 (VAS) on calcium-stimulated NO release from human umbilical vein endothelial cells exposed to oxidized LDL (oxLDL).

FIG. 14 shows the separate and combined effects of EPA, rosuvastatin (Rosuv), and VAS2780 (VAS) on calcium-stimulated ONOO⁻ release from human umbilical vein endothelial cells exposed to oxidized LDL (oxLDL).

FIG. 15 shows the separate and combined effects of EPA, rosuvastatin (Rosuv), and VAS2780 (VAS) on the ratio of NO to ONOO⁻ release from human umbilical vein endothelial cells exposed to oxidized LDL (oxLDL).

FIG. 16 shows the effects of EPA, alone or in combination with atorvastatin (Atorv) or rosuvastatin (Rosuv) on oxLDL-induced changes in HUVEC NOS3 (eNOS) levels.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

In one embodiment, the invention provides a pharmaceutical composition comprising a statin or hydroxy-derivative of a statin or pharmaceutically acceptable salt thereof and an oil comprising an omega-3 fatty acid.

### Hydroxy-Derivative of a Statin

In one embodiment, the hydroxy-derivative of a statin comprises hydroxy-atorvastatin of the following structure:

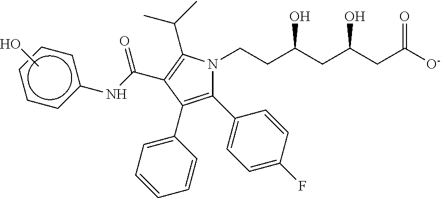

or a pharmaceutically acceptable salt thereof.

In another embodiment, the hydroxy-derivative of a statin comprises hydroxy-fluvastatin of the following structure:

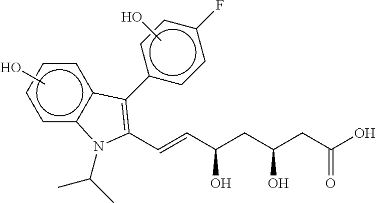

or a pharmaceutically acceptable salt thereof.

In another embodiment, the hydroxy-derivative of a statin comprises hydroxy-lovastatin, for example of the following structure:

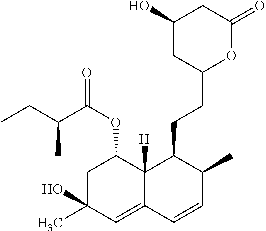 [SMILES: CC[C@H](C)C(=O)O[C@H]1C[C@](C)(O)C=C2C=C[C@H](C)[C@H](CCC3C[C@@H](O)CC(=O)O3)[C@H]21]

(6′β-hydroxy-lovastatin) or

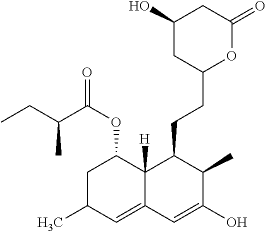 [SMILES: CC[C@H](C)C(=O)O[C@H]1CC(C)C=C2C=C(O)[C@H](C)[C@H](CCC3C[C@@H](O)CC(=O)O3)[C@H]21]

(3″-hydroxy-lovastatin) or pharmaceutically acceptable salt of either of the foregoing.

In another embodiment the hydroxy-derivative of a statin comprises hydroxy-simvastatin, for example of the following structure:

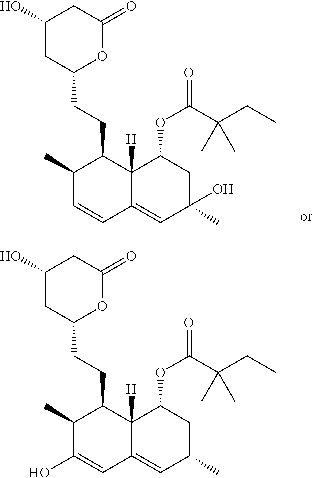

or a pharmaceutically acceptable salt of either of the foregoing.

In another embodiment the hydroxy-derivative of a statin comprises hydroxy-cerivastatin, for example of the following structure:

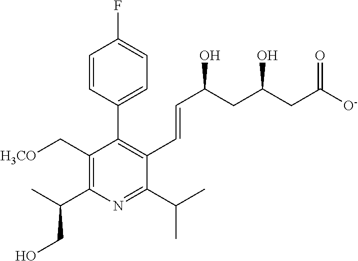 [SMILES: COCc1c([C@H](C)CO)nc(C(C)C)c(/C=C/[C@@H](O)C[C@@H](O)CC(=O)[O-])c1-c1ccc(F)cc1]

or a pharmaceutically acceptable salt thereof.

In another embodiment the hydroxy-derivative of a statin comprises hydroxy-pitavastatin, for example of the following structure:

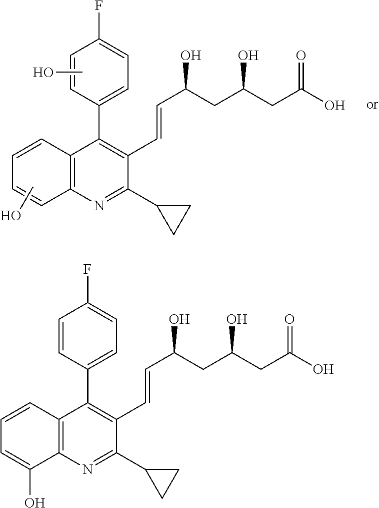

or a pharmaceutically acceptable salt of either.

In other embodiments, the hydroxy-derivative of a statin is selected from ortho or para hydroxy-atorvastatin and salts thereof, for example p-hydroxy atorvastatin calcium, p-hydroxy atorvastatin disodium, o-hydroxy atorvastatin calcium, o-hydroxy atorvastatin lactone, o-hydroxy atorvastatin-d5 calcium, o-hydroxy atorvastatin-d5 disodium, o-hydroxy atorvastatin-d5 lactone, 2-hydroxy atorvastatin bisodium, p-hydroxy atorvastatin lactone, p-hydroxy atorvastatin-d5 calcium, p-hydroxy atorvastatin-d5 lactone, and 4-hydroxy atorvastatin bisodium. In other embodiments, the statin comprises atorvastatin, simvastatin or rosuvastatin.

In various embodiments, a composition of the invention comprises a statin, hydroxy-derivative of a statin or a pharmaceutically acceptable salt thereof in an amount of about 0.01 mg to about 500 mg, about 0.1 mg to about 250 mg, or about 1 mg to about 100 mg, for example about 1 mg, about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

In various embodiments, compositions of the invention comprise an oil. In one embodiment, the oil comprises a fatty acid, for example an omega-3 fatty acid. In another embodiment, the omega-3 fatty acid comprises eicosapentaenoic acid or a pharmaceutically acceptable ester, derivative, conjugate or salt thereof, or mixtures of any of the foregoing, collectively referred to herein as “EPA.” The term “pharmaceutically acceptable” herein means that the substance in question does not produce unacceptable toxicity to the subject or interaction with other components of the composition.

In another embodiment, the oil comprises at least about 95% by weight EPA. In one embodiment, the EPA comprises all-cis eicosa-5,8,11,14,17-pentaenoic acid. In another embodiment, the EPA comprises an eicosapentaenoic acid ester. In another embodiment, the EPA comprises a C₁-C₅ alkyl ester of eicosapentaenoic acid. In another embodiment, the EPA comprises eicosapentaenoic acid ethyl ester, eicosapentaenoic acid methyl ester, eicosapentaenoic acid propyl ester, or eicosapentaenoic acid butyl ester. In yet another embodiment, the EPA comprises all-cis eicosa-5,8,11,14,17-pentaenoic acid ethyl ester.

In another embodiment, the EPA is in the form of ethyl-EPA, lithium EPA, mono-, di- or triglyceride EPA or any other ester or pharmaceutically acceptable salt of EPA, or the free acid form of EPA. The EPA may also be in the form of a 2-substituted derivative or other derivative which slows down its rate of oxidation but does not otherwise change its biological action to any substantial degree.

In another embodiment, the oil comprises docosahexaenoic acid (DHA) or a derivative thereof, for example ethyl-DHA. In another embodiment, the oil comprises at least about 95% by weight DHA or derivative thereof, for example E-DHA.

In yet another embodiment, the oil contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight, DHA or derivative thereof such as ethyl-DHA, if any. In another embodiment, a composition of the invention contains substantially no docosahexaenoic acid or derivative thereof. In still another embodiment, a composition useful in the present invention contains no docosahexaenoic acid or derivative thereof.

In one embodiment, the oil comprises ethyl eicosapentaenoate and ethyl docosahexaenoic acid in a mole ratio of about 1:1 to about 1.5:1, about 1.1:1 to about 1.4:1, for example about 1.3:1.

In another embodiment, the oil contains less than 10%, less than 9%, less than 8%, less than 7%, less than 6%, less than 5%, less than 4%, less than 3%, less than 2%, less than 1%, less than 0.5% or less than 0.25%, by weight, of any fatty acid other than EPA. Illustrative examples of a “fatty acid other than EPA” include linolenic acid (LA), arachidonic acid (AA), docosahexaenoic acid (DHA), alpha-linolenic acid (ALA), stearadonic acid (STA), eicosatrienoic acid (ETA) and/or docosapentaenoic acid (DPA). In another embodiment, an oil useful in a composition of the invention contains about 0.1% to about 4%, about 0.5% to about 3%, or about 1% to about 2%, by weight, of total fatty acids other than ethyl-EPA and/or ethyl-DHA.

In another embodiment, an oil useful in compositions of the invention has one or more of the following features: (a) eicosapentaenoic acid ethyl ester represents at least about 96%, at least about 97%, or at least about 98%, by weight, of all fatty acids present; (b) the oil contains not more than about 4%, not more than about 3%, or not more than about 2%, by weight, of total fatty acids other than eicosapentaenoic acid ethyl ester; (c) the oil contains not more than about 0.6%, not more than about 0.5%, or not more than about 0.4% of any individual fatty acid other than eicosapentaenoic acid ethyl ester; (d) the oil has a refractive index (20° C.) of about 1 to about 2, about 1.2 to about 1.8 or about 1.4 to about 1.5; (e) the composition has a specific gravity (20° C.) of about 0.8 to about 1.0, about 0.85 to about 0.95 or about 0.9 to about 0.92; (e) the oil contains not more than about 20 ppm, not more than about 15 ppm or not more than about 10 ppm heavy metals, (f) the oil contains not more than about 5 ppm, not more than about 4 ppm, not more than about 3 ppm, or not more than about 2 ppm arsenic, and/or (g) the oil has a peroxide value of not more than about 5 meq/kg, not more than about 4 meq/kg, not more than about 3 meq/kg, or not more than about 2 meq/kg.

In one embodiment, the oil comprises at least about 95% by weight ethyl eicosapentaenoate (EPA-E), about 0.2% to about 0.3% by weight ethyl octadecatetraenoate (ODTA-E), about 0.05% to about 0.20% by weight ethyl nonaecapentaenoate (NDPA-E), about 0.2% to about 0.4% by weight ethyl arachidonate (AA-E), about 0.3% to about 0.5% by weight ethyl eicosatetraenoate (ETA-E), and about 0.05% to about 0.15% ethyl heneicosapentaenoate (HPA-E).

In another one embodiment, the oil comprises at least about 96% by weight ethyl eicosapentaenoate, about 0.22% to about 0.28% by weight ethyl octadecatetraenoate, about 0.075% to about 0.15% by weight ethyl nonaecapentaenoate, about 0.25% to about 0.35% by weight ethyl arachidonate, about 0.3% to about 0.4% by weight ethyl eicosatetraenoate (ETA-E), and about 0.075% to about 0.15% ethyl heneicosapentaenoate (HPA-E).

In other embodiments, the oil comprises one or more of: (a) about 0.2% to about 0.5% by weight ethyl octadecatetraenoate, (b) about 0.05% to about 0.20% by weight ethyl nonaecapentaenoate, (c) about 0.2% to about 3% by weight ethyl arachidonate, (d) about 0.3% to about 0.5% by weight ethyl eicosatetraenoate, (e) about 0.8% to about 0.25% by weight ethyl heneicosapentaenoate, (f) about 0.02% to about 0.1% by weight ethyl 17E-icosapentaenoate, (g) about 0.02% to about 0.1% by weight ethyl 5-icosapentanoate, (h) about 0.01% to about 0.15% by weight ethyl 5E,8E-icosapentaenoate, (i) about 0.01% to about 0.15% by weight ethyl 8E,11E-icosapentaenoate, (j) about 0.01% to about 0.15% by weight ethyl 5E,14E-icosapentaenoate, (k) about 0.01% to about 0.15% by weight ethyl 5E,8E,11E,17E-icosapentaenoate, (l) no amount or substantially no amount of ethyl icosahexaenoate, (m) no amount or substantially no amount of ethyl 11Z-eicosenoate, (n) no amount or substantially no amount of ethyl docosahexaenoic acid, and/or (o) about 0.02% to about 0.1% ethyl nonadecapentaenoate. In other embodiments, the oil comprises any one or more, any two or more, any three or more, any four or more, any five or more, any six or more, any seven or more, any eight or more, any nine or more, any ten or more, any eleven or more, any twelve or more, any thirteen or more, any fourteen or more or all fifteen of: (a)-(o) immediately above.

In another embodiment, the oil comprises at least about 95% ethyl eicosapentaeoate, by weight, and about 0.2% to about 3.5% ethyl arachidonate, by weight.

In another embodiment, EPA is present in a composition of the invention in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg.

In another embodiment, a composition of the invention comprises (a) an oil, such as a fatty acid, for example an omega-3 fatty acid as described herein, and (b) a statin or a hydroxy-derivative of a statin, such as rosuvastatin and/or atorvastatin active hydroxyl metabolite (“ATM”), wherein a molar ratio of the omega-3 fatty acid(s) to the statin or hydroxy-derivative of the statin is about 1:1 to about 20:1, for example about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about or about 20:1. In some embodiments, the ratio is about 10:1. In some embodiments, EPA comprises at least about 96%, by weight of all fatty acids present in the oil (alternatively at least about 97% or at least about 98%, by weight, of all fatty acids present in the oil), and the statin or hydroxy-derivative of the statin is selected from the group consisting of: rosuvastatin, ATM, and combinations thereof. In some embodiments, the composition comprises EPA and rosuvastatin in a molar ratio of about 10:1. In some embodiments, the composition comprises EPA and ATM in a molar ratio of about 10:1.

In another embodiment, a composition of the invention is present in a capsule, for example a capsule comprising gelatin. In still another embodiment, at least about 100 mg to about 2 g of such a composition is present in each capsule.

### Therapeutic Methods

In one embodiment, the invention provides a method for treatment and/or prevention of a cardiovascular-related disease comprising administering a composition or compositions as disclosed herein to a subject in need thereof. In another embodiment the invention provides a method for treatment and/or prevention of cardiovascular-related diseases comprising co-administering to a subject in need thereof a first pharmaceutical composition comprising a hydroxy-derivative of a statin and a second pharmaceutical composition comprising an oil as set forth herein. The terms “co-administering” and “co-administration” herein includes administering two or more compositions as part of a coordinated dosing regime whether the compositions are administered sequentially, substantially simultaneously or individually.

The term “cardiovascular-related disease” herein refers to any disease or disorder of the heart or blood vessels (i.e. arteries and veins) or any symptom thereof. Non-limiting examples of cardiovascular-related diseases include hypertriglyceridemia, hypercholesterolemia, mixed dyslipidemia, coronary heart disease, vascular disease, stroke, atherosclerosis, arrhythmia, hypertension, myocardial infarction, and other cardiovascular events.

The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In one embodiment, the present invention provides a method of blood lipid therapy comprising administering to a subject or subject group in need thereof a pharmaceutical composition or compositions as described herein. In another embodiment, the subject or subject group has hypertriglyceridemia, hypercholesterolemia, mixed dyslipidemia and/or very high triglycerides.

In another embodiment, the subject or subject group being treated has a baseline triglyceride level (or mean or median baseline triglyceride level in the case of a subject group), fed or fasting, of about 200 mg/dl to about 500 mg/dl. In another embodiment, the subject or subject group has a baseline LDL-C level (or mean or median baseline LDL-C level), despite statin therapy, of about 40 mg/dl to about 100 mg/dl.

In another embodiment, the subject or subject group being treated in accordance with methods of the invention has a body mass index (BMI or mean BMI) of not more than about 45 kg/m².

In one embodiment, the subject or subject group being treated in accordance with methods of the invention exhibits a fasting baseline absolute plasma level of free total fatty acid (or mean thereof) not greater than about 300 nmol/ml, not greater than about 250 nmol/ml, not greater than about 200 nmol/ml, not greater than about 150 nmol/ml, not greater than about 100 nmol/ml, or not greater than about 50 nmol/ml.

In another embodiment, the subject or subject group being treated in accordance with methods of the invention exhibits a fasting baseline absolute plasma level of free EPA (or mean thereof in the case of a subject group) not greater than about 0.70 nmol/ml, not greater than about 0.65 nmol/ml, not greater than about 0.60 nmol/ml, not greater than about 0.55 nmol/ml, not greater than about 0.50 nmol/ml, not greater than about 0.45 nmol/ml, or not greater than about 0.40 nmol/ml. In another embodiment, the subject or subject group being treated in accordance with methods of the invention exhibits a baseline fasting plasma level (or mean thereof) of free EPA, expressed as a percentage of total free fatty acid, of not more than about 3%, not more than about 2.5%, not more than about 2%, not more than about 1.5%, not more than about 1%, not more than about 0.75%, not more than about 0.5%, not more than about 0.25%, not more than about 0.2% or not more than about 0.15%. In one such embodiment, free plasma EPA and/or total fatty acid levels are determined prior to initiating therapy.

In another embodiment, the subject or subject group being treated in accordance with methods of the invention exhibits a fasting baseline absolute plasma level of free EPA (or mean thereof) not greater than about 1 nmol/ml, not greater than about 0.75 nmol/ml, not greater than about 0.50 nmol/ml, not greater than about 0.4 nmol/ml, not greater than about 0.35 nmol/ml, or not greater than about 0.30 nmol/ml.

In another embodiment, the subject or subject group being treated in accordance with methods of the invention exhibits a fasting baseline plasma, serum or red blood cell membrane EPA level not greater than about 150 μg/ml, not greater than about 125 μg/ml, not greater than about 100 μg/ml, not greater than about 95 μg/ml, not greater than about 75 μg/ml, not greater than about 60 μg/ml, not greater than about 50 μg/ml, not greater than about 40 μg/ml, not greater than about 30 μg/ml, or not greater than about 25 μg/ml.

In another embodiment, methods of the present invention comprise a step of measuring the subject's (or subject group's mean) baseline lipid profile prior to initiating therapy. In another embodiment, methods of the invention comprise the step of identifying a subject or subject group having one or more of the following: baseline non-HDL-C value (or mean value) of about 200 mg/dl to about 400 mg/dl, for example at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl; baseline total cholesterol value (or mean value) of about 250 mg/dl to about 400 mg/dl, for example at least about 260 mg/dl, at least about 270 mg/dl, at least about 280 mg/dl or at least about 290 mg/dl; baseline vLDL-C value (or mean value) of about 140 mg/dl to about 200 mg/dl, for example at least about 150 mg/dl, at least about 160 mg/dl, at least about 170 mg/dl, at least about 180 mg/dl or at least about 190 mg/dl; baseline HDL-C value (or mean value) of about 10 to about 100 mg/dl, for example not more than about 90 mg/dl not, not more than about 80 mg/dl, not more than about 70 mg/dl, not more than about 60 mg/dl, not more than about 60 mg/dl, not more than about 50 mg/dl, not more than about 40 mg/dl, not more than about 35 mg/dl, not more than about 30 mg/dl, not more than about 25 mg/dl, not more than about 20 mg/dl, or not more than about 15 mg/dl; and/or baseline LDL-C value (or mean value) of about 30 to about 300 mg/dl, for example not less than about 40 mg/dl, not less than about 50 mg/dl, not less than about 60 mg/dl, not less than about 70 mg/dl, not less than about 90 mg/dl or not less than about 90 mg/dl.

In a related embodiment, upon treatment in accordance with the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits one or more of the following outcomes:

(a) reduced triglyceride levels compared to baseline or placebo control;

(b) reduced Apo B levels compared to baseline or placebo control;

(c) increased HDL-C levels compared to baseline or placebo control;

(d) no increase in LDL-C levels compared to baseline or placebo control;

(e) a reduction in LDL-C levels compared to baseline or placebo control;

(f) a reduction in non-HDL-C levels compared to baseline or placebo control;

(g) a reduction in vLDL levels compared to baseline or placebo control;

(h) an increase in apo A-I levels compared to baseline or placebo control;

(i) an increase in apo A-I/apo B ratio compared to baseline or placebo control;

(j) a reduction in lipoprotein a levels compared to baseline or placebo control;

(k) a reduction in LDL particle number compared to baseline or placebo control;

(l) a reduction in LDL size compared to baseline or placebo control;

(m) a reduction in remnant-like particle cholesterol compared to baseline or placebo control;

(n) a reduction in oxidized LDL compared to baseline or placebo control;

(o) a reduction in fasting plasma glucose (FPG) compared to baseline or placebo control;

(p) a reduction in hemoglobin A_{1c} (HbA_{1c}) compared to baseline or placebo control;

(q) a reduction in homeostasis model insulin resistance compared to baseline or placebo control;

(r) a reduction in lipoprotein associated phospholipase A2 compared to baseline or placebo control;

(s) a reduction in intracellular adhesion molecule-1 compared to baseline or placebo control;

(t) a reduction in interleukin-2 compared to baseline or placebo control;

(u) a reduction in plasminogen activator inhibitor-1 compared to baseline or placebo control;

(v) a reduction in high sensitivity C-reactive protein (hsCRP) compared to baseline or placebo control;

(w) an increase in serum phospholipid EPA compared to baseline or placebo control;

(x) an increase in red blood cell membrane EPA compared to baseline or placebo control; and/or

(y) a reduction or increase in one or more of serum phospholipid and/or red blood cell content of docosahexaenoic acid (DHA), docosapentaenoic acid (DPA), arachidonic acid (AA), palmitic acid (PA), staeridonic acid (SA) or oleic acid (OA) compared to baseline or placebo control.

In one embodiment, methods of the present invention comprise measuring baseline levels of one or more markers set forth in (a)-(y) above prior to dosing the subject or subject group. In another embodiment, the methods comprise administering a composition as disclosed herein to the subject after baseline levels of one or more markers set forth in (a)-(y) are determined, and subsequently taking an additional measurement of said one or more markers.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more, any 24 or more, or all 25 of outcomes (a)-(y) described immediately above.

In another embodiment, upon treatment with a composition of the present invention, the subject or subject group exhibits one or more of the following outcomes:

(a) a reduction in triglyceride level of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or mean % change) as compared to baseline or placebo control;

(b) a less than 30% increase, less than 20% increase, less than 10% increase, less than 5% increase or no increase in non-HDL-C levels or a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55% or at least about 75% (actual % change or mean % change) as compared to baseline or placebo control;

(c) an increase in HDL-C levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or mean % change) as compared to baseline or placebo control;

(d) a less than 30% increase, less than 20% increase, less than 10% increase, less than 5% increase or no increase in LDL-C levels or a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 55% or at least about 75% (actual % change or mean % change) as compared to baseline or placebo control;

(e) a decrease in Apo B levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or mean % change) as compared to baseline or placebo control;

(f) a reduction in vLDL levels of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(g) an increase in apo A-I levels of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(h) an increase in apo A-I/apo B ratio of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(i) a reduction in lipoprotein(a) levels of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(j) a reduction in mean LDL particle number of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(k) an increase in mean LDL particle size of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(l) a reduction in remnant-like particle cholesterol of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(m) a reduction in oxidized LDL of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(n) a reduction in fasting plasma glucose (FPG) of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(o) a reduction in hemoglobin A_{1c} (HbA_{1c}) of at least about 5%, at least about 10%, at least about 15%, at least about 20%, at least about 25%, at least about 30%, at least about 35%, at least about 40%, at least about 45%, or at least about 50% (actual % change or mean % change) compared to baseline or placebo control;

(p) a reduction in homeostasis model index insulin resistance of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(q) a reduction in lipoprotein associated phospholipase A2 of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(r) a reduction in intracellular adhesion molecule-1 of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(s) a reduction in interleukin-2 of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(t) a reduction in plasminogen activator inhibitor-1 of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(u) a reduction in high sensitivity C-reactive protein (hsCRP) of at least about 5%, at least about 10%, at least about 15%, at least about 20%, at least about 25%, at least about 30%, at least about 35%, at least about 40%, at least about 45%, at least about 50%, or at least about 100% (actual % change or mean % change) compared to baseline or placebo control;

(v) an increase in serum phospholipid EPA of at least about 5%, at least about 10%, at least about 15%, at least about 20%, at least about 25%, at least about 30%, at least about 35%, at least about 40%, at least about 45%, at least about 50%, at least about 100%, at least about 200% or at least about 400% (actual % change or mean % change) compared to baseline or placebo control;

(w) an increase in serum phospholipid and/or red blood cell membrane EPA of at least about 5%, at least about 10%, at least about 15%, at least about 20%, at least about 25%, at least about 30%, at least about 35%, at least about 40%, at least about 45%, r at least about 50%, at least about 100%, at least about 200%, or at least about 400% (actual % change or mean % change) compared to baseline or placebo control;

(x) a reduction or increase in one or more of serum phospholipid and/or red blood cell DHA, DPA, AA, PA and/or OA of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or mean % change) compared to baseline or placebo control; and/or

(y) a reduction in total cholesterol of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or mean % change) compared to baseline.

In one embodiment, methods of the present invention comprise measuring baseline levels of one or more markers set forth in (a)-(y) prior to dosing the subject or subject group. In another embodiment, the methods comprise administering a composition as disclosed herein to the subject after baseline levels of one or more markers set forth in (a)-(y) are determined, and subsequently taking a second measurement of the one or more markers as measured at baseline for comparison thereto.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, or all 25 or more of outcomes (a)-(y) described immediately above.

Parameters (a)-(y) can be measured in accordance with any clinically acceptable methodology. For example, triglycerides, total cholesterol, HDL-C and fasting blood sugar can be sample from serum and analyzed using standard photometry techniques. VLDL-TG, LDL-C and VLDL-C can be calculated or determined using serum lipoprotein fractionation by preparative ultracentrifugation and subsequent quantitative analysis by refractometry or by analytic ultracentrifugal methodology. Apo A1, Apo B and hsCRP can be determined from serum using standard nephelometry techniques. Lipoprotein (a) can be determined from serum using standard turbidimetric immunoassay techniques. LDL particle number and particle size can be determined using nuclear magnetic resonance (NMR) spectrometry. Remnants lipoproteins and LDL-phospholipase A2 can be determined from EDTA plasma or serum and serum, respectively, using enzymatic immunoseparation techniques. Oxidized LDL, intercellular adhesion molecule-1 and interleukin-2 levels can be determined from serum using standard enzyme immunoassay techniques. These techniques are described in detail in standard textbooks, for example Tietz Fundamentals of Clinical Chemistry, 6^{th} Ed. (Burtis, Ashwood and Borter Eds.), WB Saunders Company.

In one embodiment, a subject or subjects fast for up to 12 hours prior to blood sample collection, for example about 10 hours.

In another embodiment, the present invention provides a method of treating or preventing primary hypercholesterolemia and/or mixed dyslipidemia (Fredrickson Types IIa and IIb) in a subject in need thereof, comprising administering to the subject one or more compositions as disclosed herein. In a related embodiment, the present invention provides a method of reducing triglyceride levels in a subject or subjects when treatment with a statin or niacin extended-release monotherapy is considered inadequate (Frederickson type IV hyperlipidemia).

In another embodiment, the present invention provides a method of treating or preventing risk of recurrent nonfatal myocardial infarction in a subject with a history of myocardial infarction, comprising administering to the subject one or more compositions as disclosed herein.

In another embodiment, the present invention provides a method of treating, slowing progression of or promoting regression of atherosclerotic disease in a subject in need thereof, comprising administering to a subject in need thereof one or more compositions as disclosed herein.

In another embodiment, the present invention provides a method of inhibiting oxidation of lipopoproteins in a subject in need thereof, comprising administering to a subject in need thereof one or more compositions as disclosed herein.

In another embodiment, the present invention provides a method of scavenging free radicals in a subject in need thereof, comprising administering to a subject in need thereof one or more compositions as disclosed herein.

In another embodiment, the present invention provides a method of inhibiting metal ion chelation of lipoproteins in a subject in need thereof, comprising administering to a subject in need thereof one or more compositions as disclosed herein.

In another embodiment, the present invention provides a method of treating or preventing very high serum triglyceride levels (e.g. Types IV and V hyperlipidemia) in a subject in need thereof, comprising administering to the subject one or more compositions as disclosed herein.

In one embodiment, a composition of the invention is administered to a subject in an amount sufficient to provide a daily dose of ethyl-eicosapentaenoate of about 1 mg to about 10,000 mg, 25 about 5000 mg, about 50 to about 3000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, or about 10,000 mg.

In another embodiment, a composition of the invention is administered to a subject in an amount sufficient to provide a daily dose of hydroxy-derivative of a statin of about 0.01 mg to about 500 mg, about 0.1 mg to about 250 mg, or about 1 mg to about 100 mg, for example about 1 mg about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

In another embodiment, any of the methods disclosed herein are used in treatment of a subject or subjects that consume a traditional Western diet. In one embodiment, the methods of the invention include a step of identifying a subject as a Western diet consumer or prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example more than 50%, more than 60% or more or 70% of total calories come from these sources.

In another embodiment, any of the methods disclosed herein are used in treatment of a subject or subjects that consume less than (actual or average) about 150 g, less than about 125 g, less than about 100 g, less than about 75 g, less than about 50 g, less than about 45 g, less than about 40 g, less than about 35 g, less than about 30 g, less than about 25 g, less than about 20 g or less than about 15 g of fish per day.

In another embodiment, any of the methods disclosed herein are used in treatment of a subject or subjects that consume less than (actual or average) about 10 g, less than about 9 g, less than about 8 g, less than about 7 g, less than about 6 g, less than about 5 g, less than about 4 g, less than about 3 g, less than about 2 g per day of omega-3 fatty acids from dietary sources.

In another embodiment, any of the methods disclosed herein are used in treatment of a subject or subjects that consume less than (actual or average) about 2.5 g, less than about 2 g, less than about 1.5 g, less than about 1 g, less than about 0.5 g, less than about 0.25 g, or less than about 0.2 g per day of EPA and DHA or derivative of either from dietary sources.

In one embodiment, a composition as described herein is administered to a subject once or twice per day. In another embodiment, 1, 2, 3 or 4 capsules, each containing about 500 mg to about 1 g of a composition as described herein, are administered to a subject daily. In another embodiment, 1 or 2 capsules, each containing about 1 g of a composition as described herein, are administered to the subject in the morning, for example between about 5 am and about 11 am, and 1 or 2 capsules, each containing about 1 g of a composition as described herein, are administered to the subject in the evening, for example between about 5 pm and about 11 pm.

In another embodiment, compositions useful in accordance with methods of the invention are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example a soft gelatin capsule.

A composition for use in accordance with the invention can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In another embodiment, the invention provides use of any composition described herein for treating moderate to severe hypertriglyceridemia in a subject in need thereof, comprising: providing a subject having a fasting baseline triglyceride level of about 500 mg/dl to about 1500 mg/dl and administering to the subject a pharmaceutical composition as described herein. In one embodiment, the composition comprises about 1 g to about 4 g of eicosapentaenoic acid ethyl ester, wherein the composition contains substantially no docosahexaenoic acid.

### EXAMPLES

### Example 1

An experiment was conducted to test EPA, DHA, EPA+DHA with and without with certain statins and a statin derivative (e.g. atorvastatin, rosuvastatin, simvastatin and hydroxy-atorvastatin) in model membranes enriched with PUFAs and cholesterol at levels that reproduce disease or high CV-risk conditions (i.e. hypercholesterimia).

EPA and DHA were tested individually at a fixed concentration of 10.0 μM or in combination at 5.65 μM and 4.35 μM (EPA and DHA, respectively), which is a mole ratio of 1.3:1. Separate and combined effects of these agents on lipid peroxide (LOON) formation were examined at cholesterol-to-phospholipid (C/P) mole ratios of 0.5:1, 1.0:1 and 1.5:1. Levels of lipid hydroperoxides were also measured for EPA, DPH and EPA/DPH in cholesterol-enriched membrane prepared in the absence and presence of a statin.

1,2-Dilinoleoyl-3-sn-phosphatidylcholine (DLPC) was obtained from Avanti Polar Lipids (Alabaster, Ala.) and stored in chloroform (25 mg/ml) at −80° C. until use. Cholesterol obtained and stored in chloroform (10 mg/ml) at −20° C. CHOD-iodide color reagent (stock) was prepared according to a procedure modified from El-Saadani et al. (El-Saadani M, Esterbauer H, El-Sayed M, Goher M, Nassar A Y, Jurgens G. A spectrophotometric assay for lipid peroxides in serum lipoproteins using commercially available reagent. J Lipid Res 1989; 30:627-30) consisted of 0.2 M K₂HPO₄, 0.12 M KI, 0.15 mM NaN₃, 10 μM ammonium molybdate, and 0.1 g/L benzalkonium chloride. Prior to experimental use, the CHOD reagent was activated by adding 24 μM ethylenediaminetetraacetic acid (EDTA), 20 μM butylated hydroxytoluene (BHT), and 0.2% Triton X-100. The statin was prepared in ethanol just prior to experimental use and added together with component lipids containing fixed amounts of EPA, DPH or EPA/DPH at equimolar levels. The compounds and lipids were added in combination during membrane sample preparation to ensure full incorporation into the lipid bilayers.

Membrane samples consisting of DLPC±cholesterol, with cholesterol-to-phospholipid (C/P) mole ratios ranging from 0.5 to 1.5, were prepared as follows. Component lipids (in chloroform) were transferred to 13×100 mm test tubes and shell-dried under a steady stream of nitrogen gas while vortex mixing. The lipid was co-dried with EPA, DPH or EPA/DPH prepared in the absence or presence of a statin at equimolar levels.

Residual solvent was removed by drying for a minimum of 3 h under vacuum. After desiccation, each membrane sample was resuspended in diffraction buffer (0.5 mM HEPES, 154 mM NaCl, pH 7.3) to yield a final phospholipid concentration of 1.0 mg/ml. Multilamellar vesicles (MLV) were formed by vortex mixing for 3 minutes at ambient temperature. Bangham A D, Standish M M, Watkins J C. Diffusion of univalent ions across the lamellae of swollen phospholipids. J Mol Biol 1965; 13:238-52. Immediately after initial MLV preparation, aliquots of each membrane sample will be taken for baseline (0 h) peroxidation analyses.

All lipid membrane samples were subjected to time-dependent autoxidation by incubating at 37° C. in an uncovered, shaking water bath. Small aliquots of each sample were removed at 24 h intervals and combined with 1.0 ml of active CHOD-iodide color reagent. To ensure spectrophotometric readings within the optimum absorbance range, sample volumes taken for measurement of lipid peroxide formation were adjusted for length of peroxidation and range between 100 and 10 μl. Test samples were immediately covered with foil and incubated at room temperature for >4 h in the absence of light. Absorbances were measured against a CHOD blank at 365 nm using a Beckman DU-640 spectrophotometer.

The CHOD colorimetric assay is based on the oxidation of iodide (I⁻) by lipid hydroperoxides (LOON) and proceeds according to the following reaction scheme:

LOOH+2H⁺+3I→LOH+H₂O+I₃⁻

The quantity of triiodide anion (I₃⁻) liberated in this reaction is directly proportional to the amount of lipid hydroperoxides present in the membrane sample. The molar absorptivity value (ε) of I₃⁻ is 2.46×10⁴ M⁻¹ cm⁻¹ at 365 nm.

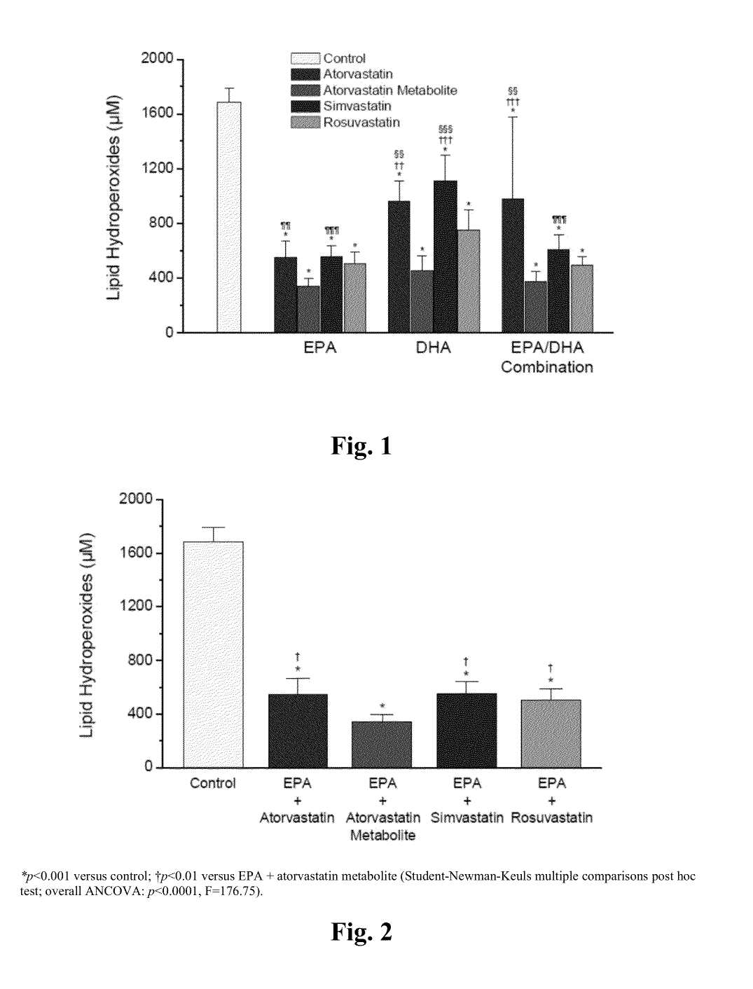

As is shown in FIG. 1, EPA, DHA and EPA/DHA plus hydroxy-atorvastatin exhibited significantly lower lipid hydroperoxide formation compared to control.

As shown in FIG. 2, EPA plus hydroxy-atorvastatin exhibited significantly lower lipid hydroperoxide formation compared to EPA+atorvastatin, EPA+simvastatin or EPA+rosuvastatin.

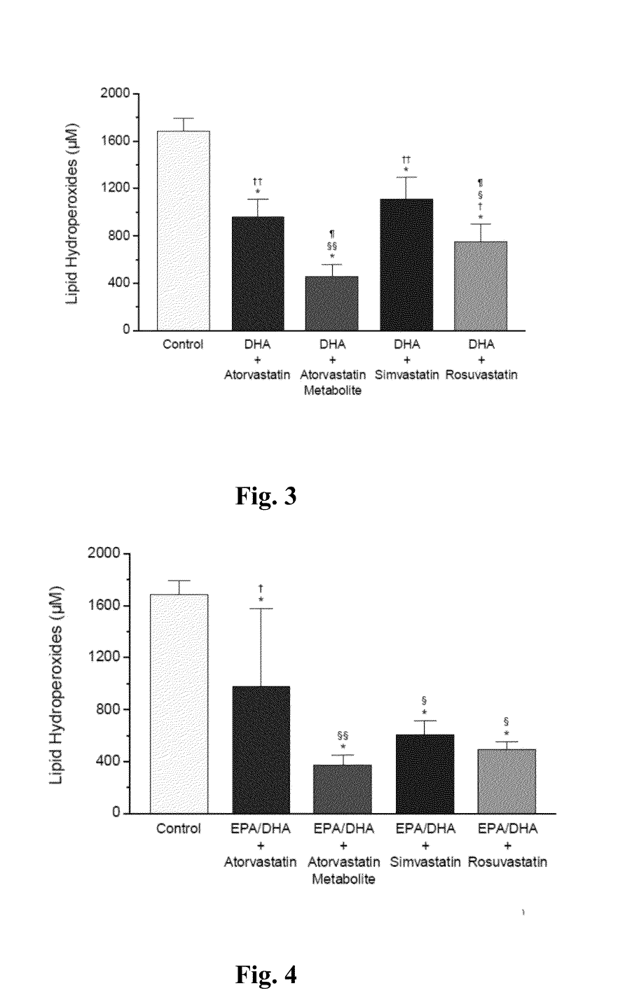

As shown in FIG. 3, DHA plus hydroxy-atorvastatin exhibited significantly lower lipid hydroperoxide formation compared to DHA+atorvastatin, DHA+simvastatin or DHA+rosuvastatin.

As shown in FIG. 4, EPA/DHA plus hydroxy-atorvastatin exhibited significantly lower lipid hydroperoxide formation than EPA/DHA plus atorvastatin.

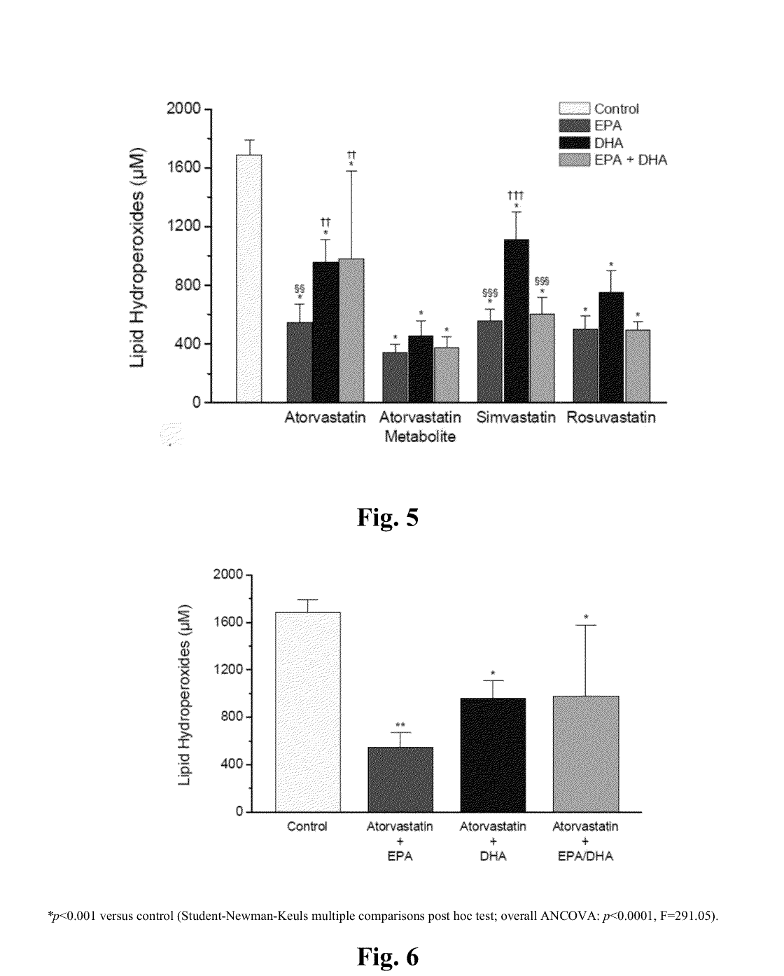

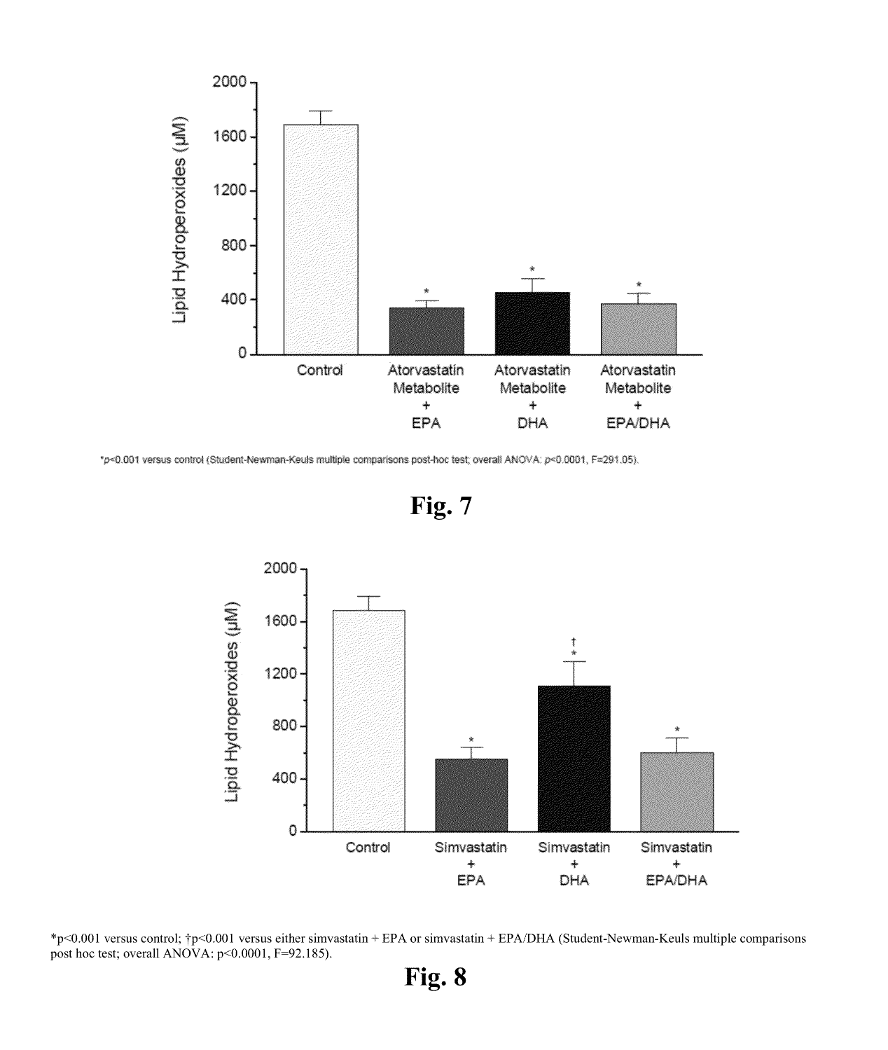

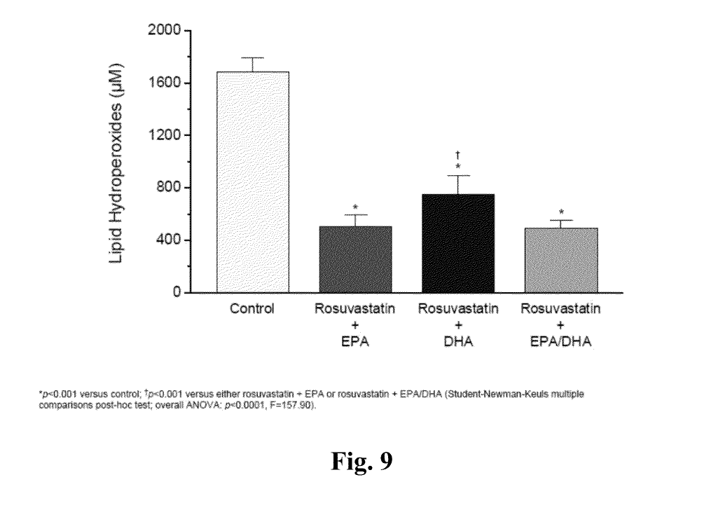

As shown in FIG. 5, EPA, DHA and EPA/DHA plus hydroxy-atorvastatin or rosuvastatin exhibited significantly lower lipid hydroperoxide formation compared to control.

### Example 2

An experiment was conducted to test the effects of EPA with rosuvastatin or atorvastatin active o-hydroxyl metabolite (“ortho-hydroxy atorvastatin” or “ATM”) on nitric oxide (NO) and peroxynitrite (ONOO⁻) release by human umbilical vein epithelial cells (“HUVECs”) following stimulation with oxidized LDL. Nitric oxide bioavailability, peroxynitrite bioavailability, and the ratio of bioavailable nitric oxide to bioavailable peroxynitrite (NO/ONOO⁻) serve as useful indicators of endothelial cell function. In particular, the NO/ONOO⁻ ratio can serve as an indicator of normal epithelial cell function.

In this experiment, HUVECs were equipped with prophyrinic nanosensors and then treated with calcium ionophore (1.0 μM). The HUVECs were then exposed to a solution of oxidized LDL (10 mg/dL). Treatment with oxLDL caused NO levels to decrease by 60% (to 156+/−18 nM) and ONOO⁻ levels to increase by 38% (to 283+/−16 nM) compared to untreated cells (386+/−29 nM NO and 205+/−31 nM ONOO⁻).

HUVECs exposed to oxLDL were then treated with EPA alone (10.0 μM), a combination of EPA (10.0 μM) and rosuvastatin (1.0 μM), or a combination of EPA (10.0 μM) and ATM (1.0 μM). Results are shown in Table 1, below.

TABLE 1

| Entry | EPA | Rosuvastatin | ATM | NO (nM) | ONOO⁻(nM) | NO/ONOO⁻ |
|---|---|---|---|---|---|---|
| 1: control | 0 | 0 | 0 | 386 ± 29 | 205 ± 31 | 1.9 |
| 2: oxLDL | 0 | 0 | 0 | 156 ± 18 | 283 ± 16 | 0.5 |
| 3 | 10.0 μM | 0 | 0 | 208 ± 32 | 203 ± 21 | 1.0 |
| 4 | 0 | 1.0 μM | 0 | 191 ± 33 | 252 ± 39 | 0.8 |
| 5 | 0 | 0 | 1.0 μM | 199 ± 20 | 236 ± 35 | 0.8 |
| 6 | 10.0 μM | 1.0 μM | 0 | 221 ± 39 | 174 ± 34 | 1.7 |
| 7 | 10.0 μM | 0 | 1.0 μM | 250 ± 23 | 174 ± 34 | 1.3 |

As shown in Table 1 above, the combination of EPA and rosuvastatin (entry 6) or ATM (entry 7) improved NO bioavailability beyond that expected from the combination of the component parts (entries 3, 4, 5 compared to entry 2). These data indicated that there are novel unexpected interactions between EPA and statins that may reduce CV risk beyond their known effect on lipid levels.

### Example 3

An experiment was conducted to test the effects of EPA with rosuvastatin, atorvastatin or o-hydroxyatorvastatin (an active hydroxyl metabolite, “ATM”) on nitric oxide (NO) and peroxynitrite (ONOO⁻) release by human umbilical vein epithelial cells (“HUVECs”) following stimulation with oxidized LDL. Nitric oxide bioavailability, peroxynitrite bioavailability, and the ratio of bioavailable nitric oxide to bioavailable peroxynitrite (NO/ONOO⁻) serve as useful indicators of endothelial cell function. In particular, the NO/ONOO⁻ ratio can serve as an indicator of normal epithelial cell function.

Human umbilical vein endothelial cells (HUVECs) were isolated into primary cultures from female donors by Clonetics (San Diego, Calif.) and purchased as proliferating cells. All cell culture donors were healthy, with no pregnancy or prenatal complications. The cultured cells were incubated in 95% air/5% CO₂ at 37° C. and passaged by an enzymatic (trypsin) procedure. The confluent cells (4 to 5×10⁵ cells/35 mm dish) were placed with minimum essential medium containing 3 mM L-arginine and 0.1 mM BH₄ [(6R)-5,6,7,8-tetrahydrobiopterin]. Before experimental use, the cells (from second or third passage) were rinsed twice with Tyrode-HEPES buffer with 1.8 mM CaCl₂.

The effects of EPA treatment were tested in the absence and presence of statins in HUVECs following exposure to oxidized LDL (oxLDL). The oxLDL causes eNOS uncoupling and endothelial dysfunction to reproduced disease-like conditions. Venous blood from healthy normolipidemic volunteers was collected into Na-EDTA (1 mg/mL blood) vacuum tubes after a 12-hour fast. Plasma was immediately separated by centrifugation at 3,000 g for 10 minutes at 4° C. LDL (δ=1.020 to 1.063 g/mL) was separated from freshly drawn plasma by preparative ultracentrifugation with a Beckman ultracentrifuge equipped with an SW-41 rotor.¹² The density of plasma was adjusted to 1.020 g/mL with sodium chloride solution, the plasma was centrifuged at 150,000 g for 24 hours, and the chylomicron-rich layers were discarded. The remaining fraction, after adjustment of density at 1.063 g/mL with potassium bromide, was centrifuged at 150,000 g for 24 hours to isolate LDL from the HDL fraction. The purified LDL was dialyzed for 96 hours against PBS containing 0.3 mM EDTA at 4° C., then stored at 4° C.

The purified LDL was oxidized according to the methods of Huber et al. (Free Radic. Res. Commun. 1990; 8(3):167-173). A sample of LDL was dialyzed against Tris/NaCl Buffer (50 mM Tris in 0.15 M NaCl, pH 8.0) to remove the EDTA. Tris-NaCl buffer was added to the dialyzed n-LDL to adjust the protein concentration to 30 mg/mL. A 1-mL aliquot of 20 μM CuSO₄ was added to 1 mL of dialyzed normal LDL. Oxidation at 37° C. was followed spectrophotometrically (234 nm) over a period of 24 hours until oxidation was complete. The oxLDL was then dialyzed at 4° C. with 4 L of Tris buffer, filtered with a 0.22 μm filter, and stored under nitrogen at 4° C.

Oxidation was monitored by the use of measurements of TBARS. Briefly, LDL was incubated with thiobarbituric acid (0.5 wt/vol, in H₂SO₄, 50 mM) for 30 minutes at 100° C. The solution then was centrifuged for 5 minutes, and the difference in absorbency at 532 and 580 nm was calculated. TBARS concentration was determined as malondialdehyde (MDA) equivalents with the use of an MDA standard curve.

Concurrent measurements of NO and ONOO⁻ were performed with tandem electrochemical nanosensors combined into one working unit with a total diameter of 200-400 nm. Their design was based on previously developed and chemically modified carbon-fiber technology. Each of the nanosensors was made by depositing a sensing material on the tip of a carbon fiber (length 4 to 5 μm, diameter 100-200 nm). The fibers were sealed with nonconductive epoxy and electrically connected to copper wires with conductive silver epoxy. Conductive films of polymeric Ni(II) tetrakis(3-methoxy-4-hydroxyphenyl)porphyrin and Mn(III) [2.2]paracyclophanylporphyrin were used for the NO and ONOO⁻ sensors, respectively.

The amperometric method (with a response time of 0.1 ms) provides a quantitative signal (current) that is directly proportional to changes (from basal levels) in NO or ONOO⁻ concentration. Amperometric measurements were performed with a Gamry III double-channel potentiostat. Basal NO or ONOO⁻ levels were measured by differential pulse voltammetry in separate experiments.

All measurements of NO and ONOO⁻ were performed on intact endothelial cells. The NO/ONOO⁻ nanosensor module was positioned 5±2 μm from the surface of each individual endothelial cell using a computer-controlled M3301 micromanipulator (x-y-z resolution of 0.2 μm) and microscope (both from World Precision Instruments, Berlin, Germany) fitted with a CD camera. After establishing a background current, EPA, in the absence and presence of different statins, was added to the cells. Rapid changes in current (proportional to the molar concentrations of NO or ONOO⁻ released) were observed after the addition of Cal and were monitored continuously.

Primary human umbilical vein endothelial cells (HUVECs) were incubated with vehicle or oxidized human low density lipoprotein (oxLDL) for 20 min prior to treatment with EPA and/or statins. After this incubation period, the cells were treated vehicle or with 10 μM EPA in the absence or presence of either 1.0 μM rosuvastatin calcium salt or 1.0 μM atorvastatin hydroxyl metabolite (ATM) for 1 hour. Endothelial basal media was used for all the treatments. Controls were supplied with an equivalent volume of endothelial basal media.

Subunits of nitric oxide synthase (NOS3) were measured by real-time quantitative PCR (qPCR) using an Applied Biosystems StepOnePlus Real-Time PCR system. Beta actin (ACTB) was quantitated and used as the housekeeping gene. DNA-free RNA was isolated from the HUVEC preparations. An equal quantity of total RNA from each sample was reverse transcribed. An equal quantity of total cDNA from each sample was then used to perform qPCR with a predesigned Applied Biosystems TaqMan gene expression assay for NOS3 (eNOS). The expression of eNOS, within each sample, was normalized against ACTB expression and expressed relative to the vehicle. For amplifying the gene sequenced, PCR was carried out with a first step at 95° C. for 10 min (first step) and then 40 cycles of 95° C. for 15 seconds and 60° C. for 1 min, plus a final incubation step at 72° C. for 10 min.

Data are presented as mean±S.D. for (N) separate samples or experiments. Differences between groups were analyzed using the two-tailed, Student t-test (for comparisons between only two groups) or ANOVA followed by Student-Newman-Keuls multiple comparisons post-hoc analysis (for comparisons between three or more groups). Only differences with probability values less than 0.05 were considered significant.

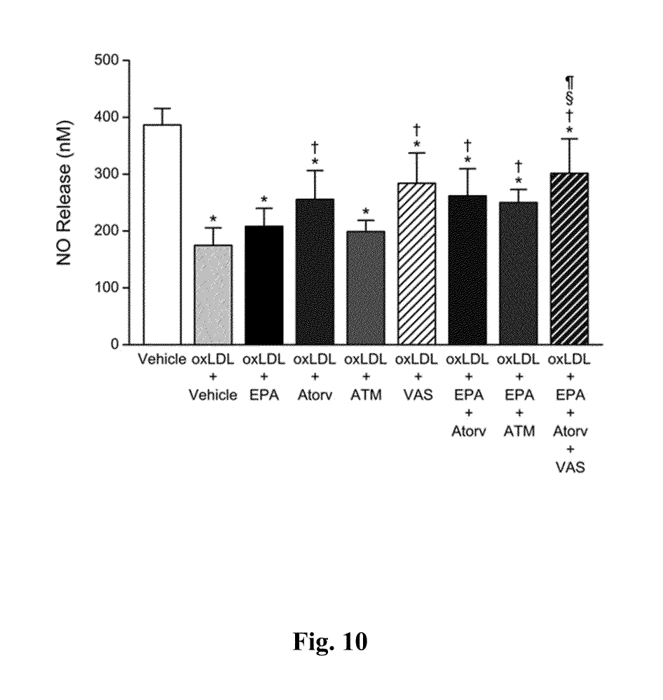

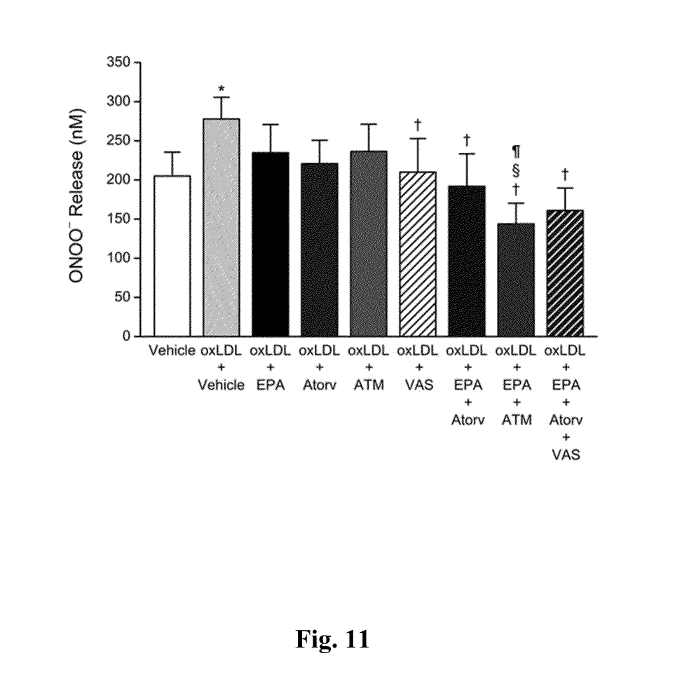

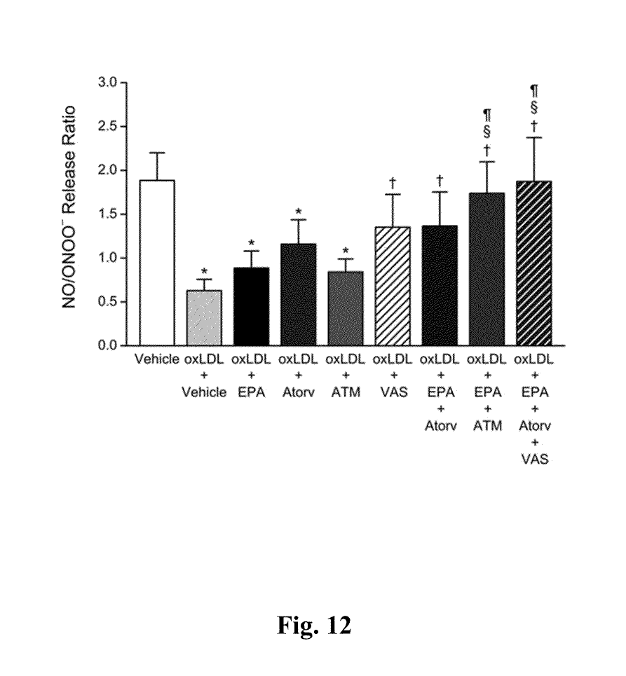

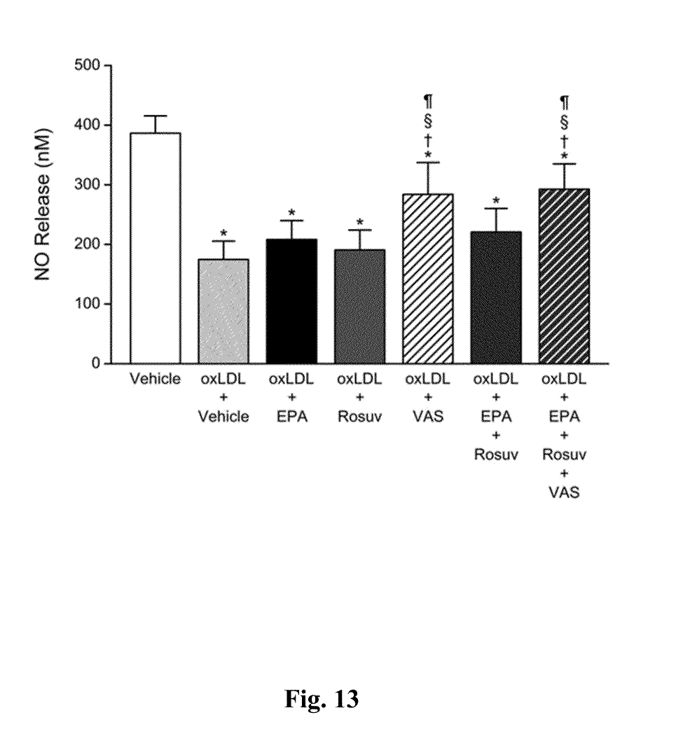

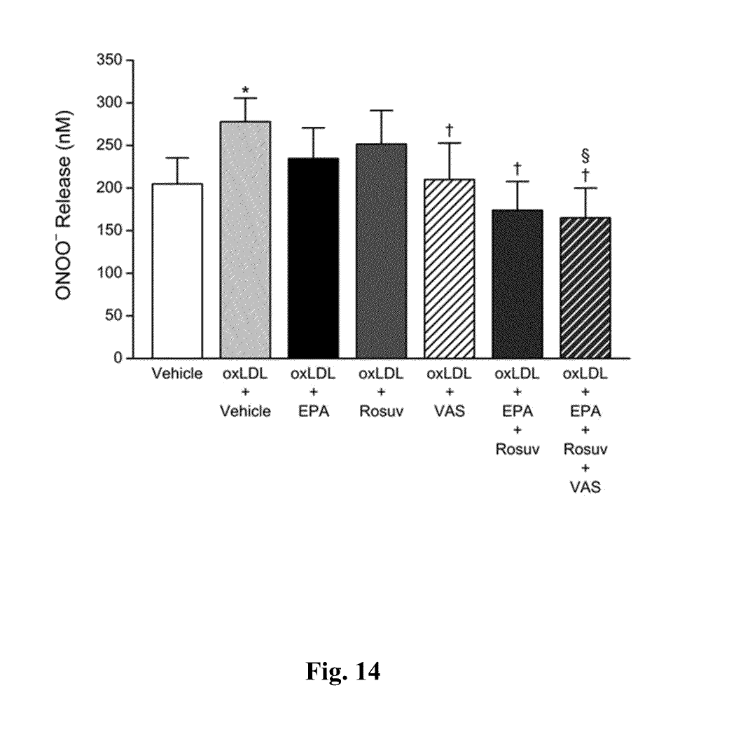

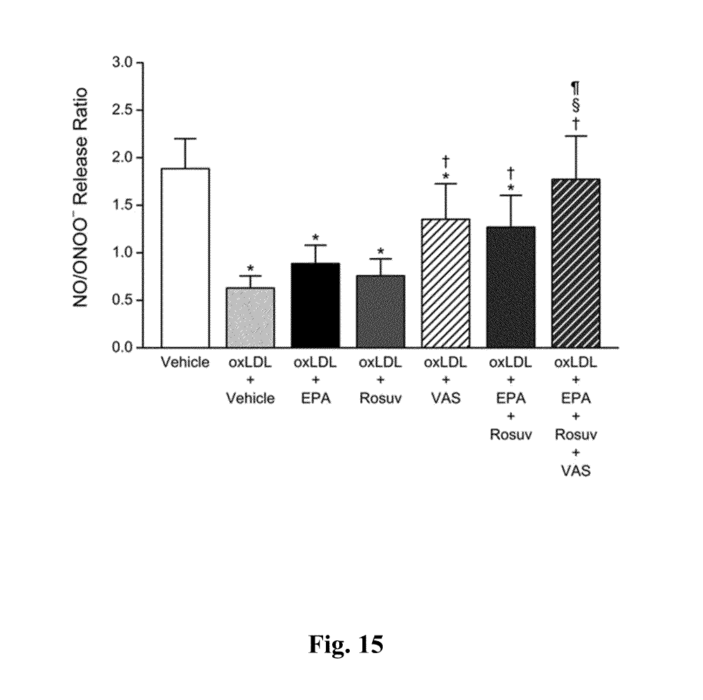

The separate versus combined effects of EPA and statins on NO and ONOO⁻ release are summarized in FIGS. 10-15. In FIGS. 10-12, the interactions of EPA with atorvastatin and ATM are summarized while FIGS. 13-15 review the results of EPA with rosuvastatin. As evidenced in all of the figures, exposure of cells to oxLDL decreased NO release from HUVECs by 55% (386±29 nM to 175±31 nM) and increased ONOO⁻ release by 36% (205±31 nM to 278±28 nM) as compared to untreated cells (p<0.01). The NO/ONOO⁻ ratio, an indicator of normal EC function, decreased by 76% with oxLDL treatment. In ECs exposed to oxLDL, treatment with EPA alone increased NO release by 18% (208±32 nM) and reduced ONOO⁻ release by 16% (203±21 nM). The NO/ONOO⁻ ratio was increased by 41% with EPA treatment alone in HUVECs.

The addition of statins separately increased the ratio of NO/ONOO⁻ in these cells by 21% (rosuvastatin), 33% (ATM) and 84% (atorvastatin) at a concentration of 1.0 μM. The combination of EPA with the statins produced the most potent effects on NO release. In these cells, addition of EPA with statins dramatically increased the NO/ONOO⁻ ratio by 201% (EPA and rosuvastatin), 276% (EPA and ATM) and 217% (EPA and atorvastatin). The NADPH oxidase inhibitor, VAS-2780, separately increased the NO/ONOO⁻ ratio by two-fold and this benefit was further enhanced when further treated with EPA in combination with rosuvastatin or atorvastatin. For rosuvastatin and ATM, the increase in the NO/ONOO⁻ ratio became significant only when these statins were tested in combination with EPA.

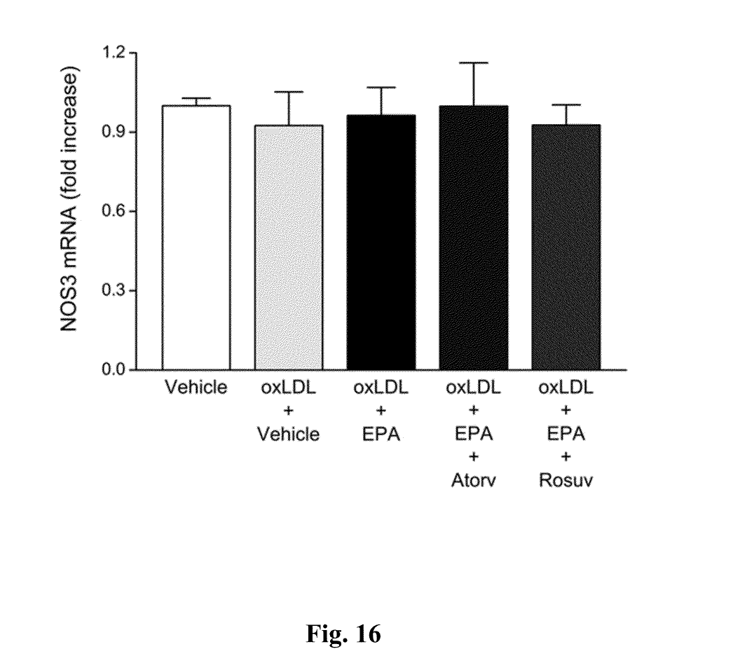

We also measured changes in eNOS expression by qPCR in HUVECs as a function of treatment. As compared to vehicle, there were no changes in eNOS expression with EPA in the absence or presence of either rosuvastatin or atorvastatin (FIG. 16).

What is claimed is:

1. A pharmaceutical composition comprising
(a) a hydroxy-derivative of a statin or pharmaceutically acceptable salt thereof and
(b) an oil comprising an omega-3 fatty acid, wherein a molar ratio of the omega-3 fatty acid to the hydroxy-derivative of the statin is about 1:1 to about 20:1.

2. The pharmaceutical composition of claim 1 wherein the omega-3 fatty acid comprises ethyl eicosapentaenoate.

3. The pharmaceutical composition of claim 1 wherein the oil comprises at least 95% by weight ethyl eicosapentaenoate.

4. The pharmaceutical composition of claim 1 wherein the oil comprises at least 95% by weight ethyl docosahexaenoic acid.

5. The pharmaceutical composition of claim 3 wherein the oil comprises ethyl eicosapentaenoate and ethyl docosahexaenoic acid.

6. The pharmaceutical composition of claim 5 wherein the oil comprises ethyl eicosapentaenoate and ethyl docosahexaenoic acid in a mole ratio of about 1:1 to about 1.5:1.

7. The pharmaceutical composition of claim 1 wherein the hydroxy-derivative of a statin or pharmaceutically acceptable salt thereof is selected from a hydroxy-derivative of atorvastatin, mevastatin, pitavastatin, pravastatin, rosuvastatin, fluvastatin, simvastatin, lovastatin, cerivastatin and pharmaceutically acceptable salts thereof,

8. The pharmaceutical composition of claim 7 wherein the hydroxy-derivative of a statin is selected from ortho or para hydroxy-atorvastatin and pharmaceutically acceptable salts thereof.

9. The pharmaceutical composition of claim 8 wherein the hydroxy-derivative of a statin or pharmaceutically acceptable salt thereof is selected from the group consisting of: o-hydroxy atorvastatin, p-hydroxy atorvastatin, p-hydroxy atorvastatin calcium, p-hydroxy atorvastatin disodium, o-hydroxy atorvastatin calcium, o-hydroxy atorvastatin lactone, o-hydroxy atorvastatin-d5 calcium, o-hydroxy atorvastatin-d5 disodium, o-hydroxy atorvastatin-d5 lactone, 2-hydroxy atorvastatin bisodium, p-hydroxy atorvastatin lactone, p-hydroxy atorvastatin-d5 calcium, p-hydroxy atorvastatin-d5 lactone, and 4-hydroxy atorvastatin bisodium.

10. The pharmaceutical composition of claim 1 further comprising tocopherol in an amount of about 0.1% to about 0.3%, by weight.

11. The pharmaceutical composition of claim 1 wherein about 0.5 g to about 2 g of said composition is present in a capsule shell.

12. The pharmaceutical composition of claim 1, wherein the molar ratio of the omega-3 fatty acid to the hydroxy-derivative of the statin is about 10:1.

13. The pharmaceutical composition of claim 3, wherein the hydroxy-derivative of a statin is a hydroxy-atorvastatin or a pharmaceutically acceptable salt thereof.

14. The pharmaceutical composition of claim 4, wherein the hydroxy-derivative of a statin is a hydroxy-atorvastatin or a pharmaceutically acceptable salt thereof.

15. A pharmaceutical composition comprising (a) rosuvastatin and (b) an oil comprising at least 95% by weight ethyl eicosapentaenoate, wherein a molar ratio of ethyl eicosapentaenoate to rosuvastatin is about 1:1 to about 20:1.

16. The pharmaceutical composition of claim 15, wherein the molar ratio is about 10:1.

17. A pharmaceutical composition comprising (a) rosuvastatin and (b) an oil comprising at least 95% by weight ethyl docosahexaenoic acid, wherein a molar ratio of ethyl docosahexaenoic acid to rosuvastatin is about 1:1 to about 20:1.

18. The pharmaceutical composition of claim 17, wherein the molar ratio is about 10:1.

19. The composition of claim 1, wherein the hydroxy-derivative of a statin is ortho-hydroxy atorvastatin.

20. A method of treating a cardiovascular-related disease in a subject in need thereof comprising administering to the subject the composition of claim 1.

21. The method of claim 20, wherein the cardiovascular disease is selected from hypertriglyceridemia, hypercholesterolemia, mixed dyslipidemia, coronary heart disease, vascular disease, stroke, atherosclerosis, arrhythmia, hypertension, and myocardial infarction.

22. The method of claim 20, wherein the cardiovascular disease is atherosclerosis.

23. A method of treating a cardiovascular-related disease in a subject in need thereof comprising administering to the subject the composition of claim 13.

24. A method of treating a cardiovascular-related disease in a subject in need thereof comprising administering to the subject the composition of claim 14.

25. A method of treating a cardiovascular-related disease in a subject in need thereof comprising administering to the subject the composition of claim 15.

26. A method of treating a cardiovascular-related disease in a subject in need thereof comprising administering to the subject the composition of claim 17.
